(19) United States
(12) Patent Application Publication
(10) Pub. No.: US 2019/0209401 A1
(43) Pub. Date: Jul. 11, 2019

(54) Bandage Administering Assembly

(71) Applicants: Frank Queen, Hallendale Beach, FL (US); Gustavo Bugallo, Hallandale Beach, FL (US)

(72) Inventors: Frank Queen, Hallendale Beach, FL (US); Gustavo Bugallo, Hallandale Beach, FL (US)

(21) Appl. No.: 15/866,644

(22) Filed: Jan. 10, 2018

(51) Int. Cl. A61F 15/00; A61F 15/02

(52) U.S. Cl. CPC A61F 15/002; A61F 13/00072; A61F 15/02

(57) ABSTRACT

A bandage administering assembly for releasing a selected amount of bandage includes a grip and a mount that is attached to the grip. A roller is rotatably mounted on the mount. A bandage is wound up to define a bandage roll and the bandage roll is positioned on the roller. A free end of the bandage roll is pulled to unwind the bandage roll from the roller. A dispenser is attached to the mount. The bandage is extended through the dispenser to define an exposed portion of the bandage. The dispenser is used to selectively cut off the exposed portion.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

### (2) Description of Related Art Including Information Disclosed Under 37 CFR 1.97 and 1.98

The disclosure and prior art relates to bandage releasing devices and more particularly pertains to a new bandage releasing device for releasing a selected amount of bandage.

### BRIEF SUMMARY OF THE INVENTION

An embodiment of the disclosure meets the needs presented above by generally comprising a grip and a mount that is attached to the grip. A roller is rotatably mounted on the mount. A bandage is wound up to define a bandage roll and the bandage roll is positioned on the roller. A free end of the bandage roll is pulled to unwind the bandage roll from the roller. A dispenser is attached to the mount. The bandage is extended through the dispenser to define an exposed portion of the bandage. The dispenser is used to selectively cut off the exposed portion.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a front side view of a bandage administering assembly according to an embodiment of the disclosure.

FIG. 2 is a front side view of an embodiment of the disclosure.

FIG. 3 is a bottom view of an embodiment of the disclosure.

FIG. 4 is a back view of an embodiment of the disclosure.

FIG. 5 is a front view of an embodiment of the disclosure.

FIG. 6 is a side view of an embodiment of the disclosure.

FIG. 7 is a cross-sectional view of an embodiment of the disclosure taken along line 7-7 of FIG. 5.

### DETAILED DESCRIPTION OF THE INVENTION

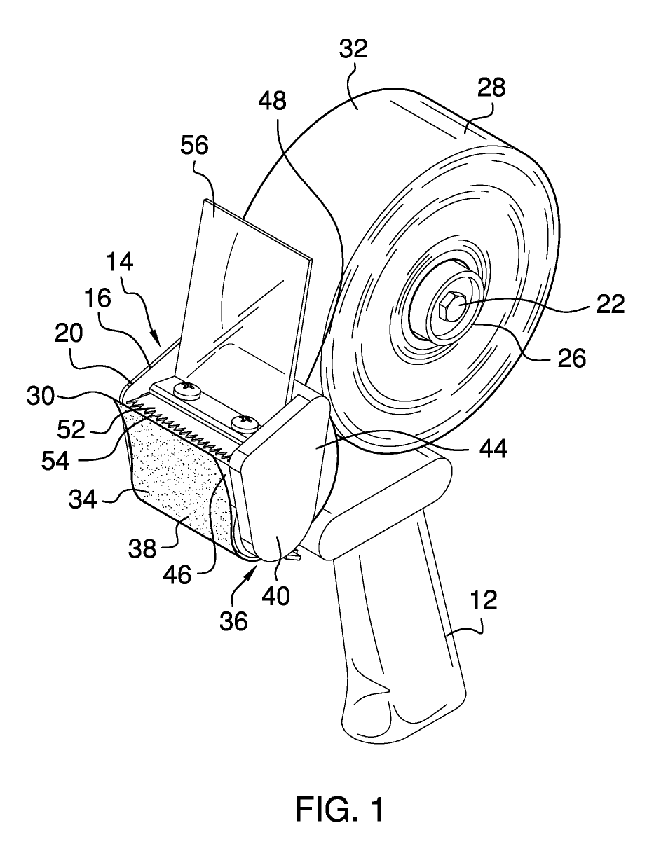

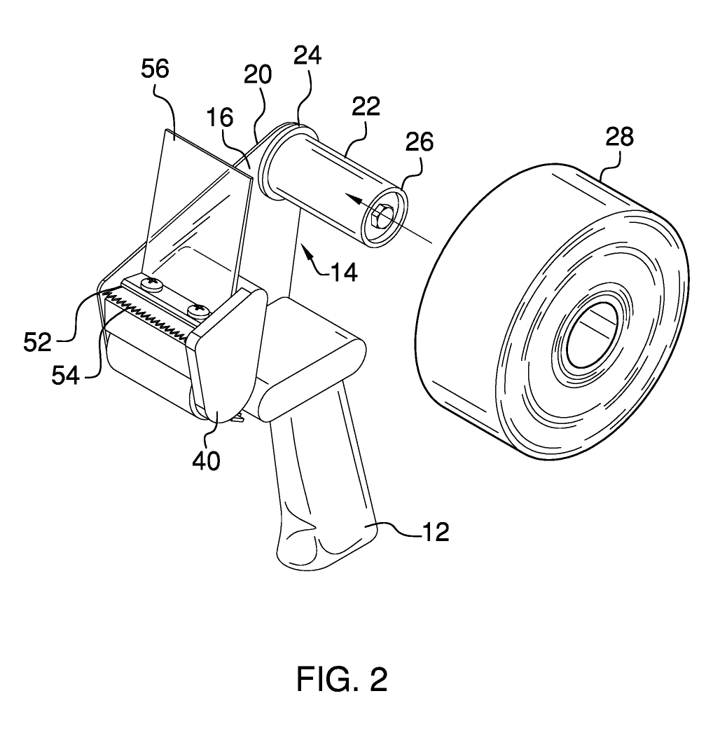

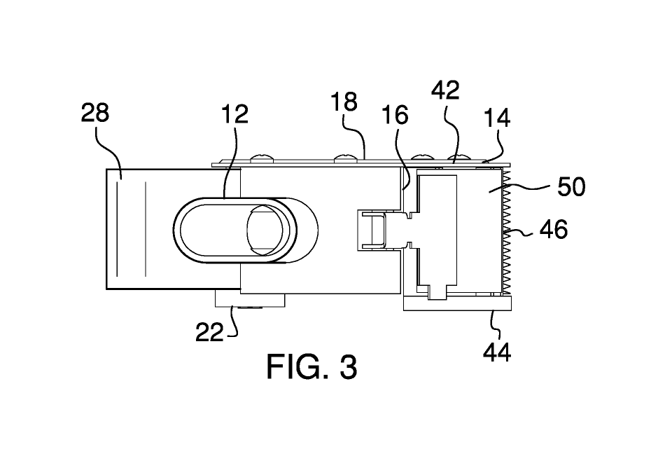

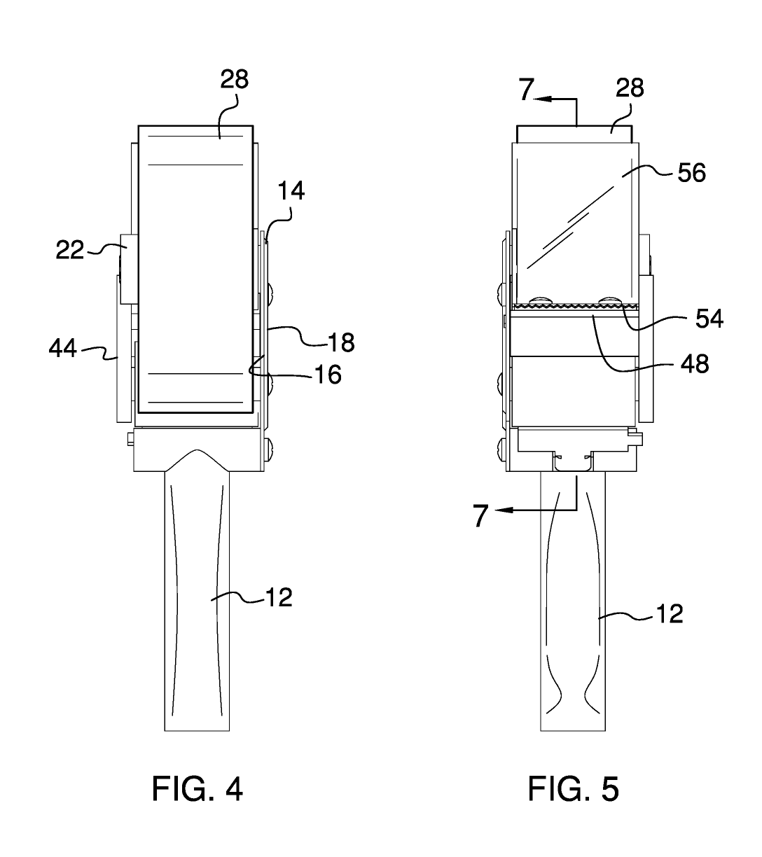

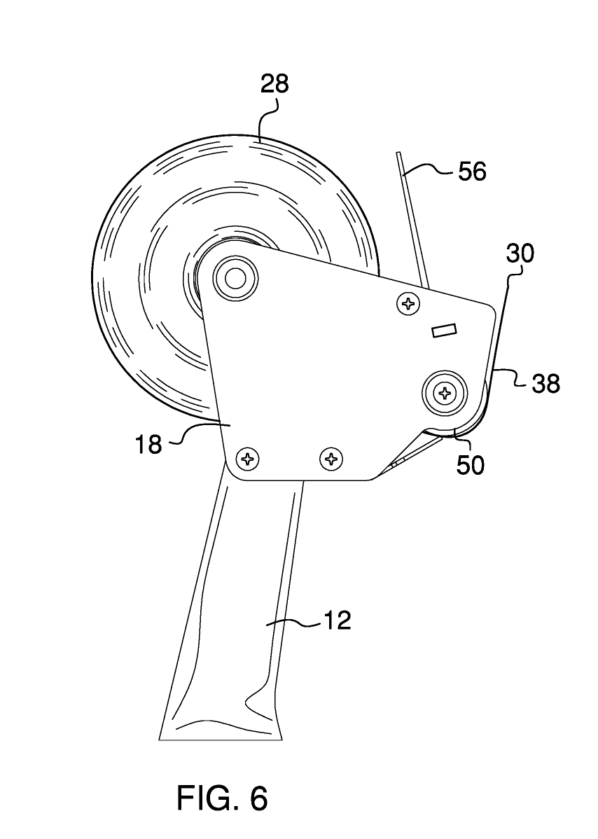

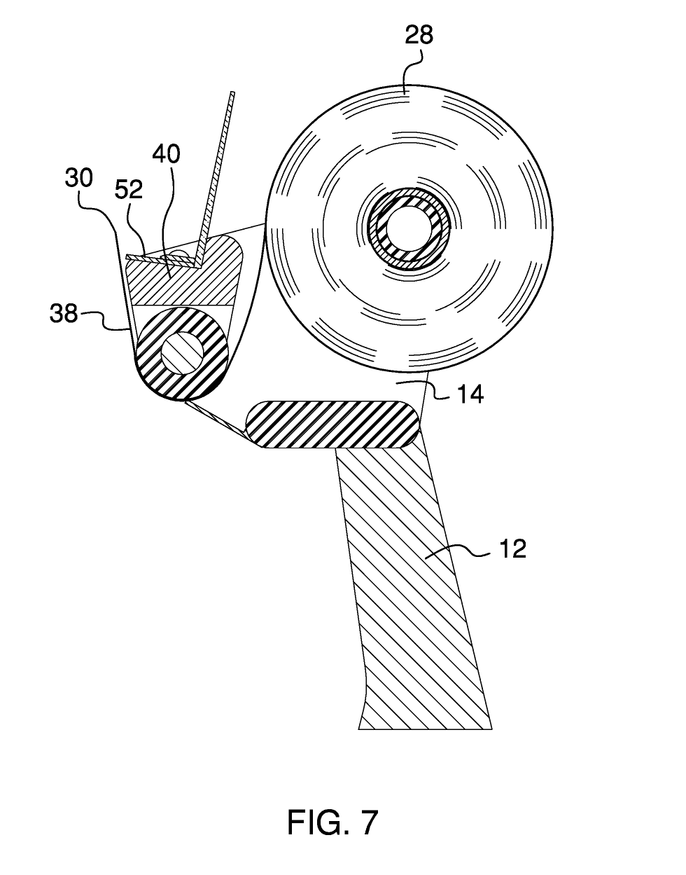

With reference now to the drawings, and in particular to FIGS. 1 through 7 thereof, a new bandage releasing device embodying the principles and concepts of an embodiment of the disclosure and generally designated by the reference numeral 10 will be described.

As best illustrated in FIGS. 1 through 7, the bandage administering assembly 10 generally comprises a grip 12 and a mount 14 that is attached to the grip 12. The mount 14 has a first surface 16, a second surface 18 and a perimeter edge 20 that extends between and is attached to the first surface 16 and the second surface 18.

A roller 22 is rotatably mounted on the mount 14. The roller 22 has a first lateral edge 24 and a second lateral edge 26 wherein the first lateral edge 24 is rotatably coupled to the first surface 16 of the mount 14. The second lateral edge 26 extends outwardly from the mount 14. A bandage 28 is wound up to define a bandage roll 28. The bandage roll 28 is positioned on the roller 22 and a free end 30 of the bandage roll 28 is pulled to unwind the bandage roll 28 from the roller 22. The bandage 28 comprises a cloth 32 that has an adhesive 34 positioned thereon. The adhesive abuts the cloth 32 when the bandage 28 is wound up.

A dispenser 36 is attached to the mount 14. The bandage 28 is extended through the dispenser 36 to define an exposed portion 38 of the bandage 28. The dispenser 36 is used to selectively cut off the exposed portion 38. The dispenser 36 comprises a block 40 is attached to and extends outwardly from the mount. The block 40 guides the bandage 28 outwardly from the roller 22. The adhesive 34 of the bandage 28 faces away from the block 40 when pulled outwardly from the roller 22. The block 40 has a first lateral side 42, a second lateral side 44, a front side 46, a top side 48 and a bottom side 50. The first lateral side 42 is coupled to the first surface 16 of the mount 14.

A cutting member 50 is attached to the top side 48 of the block 40 and is used to cut the bandage 28. The bandage 28 is extended beneath the bottom side 50 of the block 40 and upwardly along the front side 46 of the block 50 to define an exposed portion of the bandage 38. The cutting member 52 is aligned with the front side 46 of the block 40 and has teeth 54 that extend outwardly of the front side 46 of the block 40. The teeth 54 cut the exposed portion 38 of the bandage 28 to a selected length.

A window 56 is attached to and extends outwardly from the block 40. The window 56 prevents the exposed portion 38 of the bandage 28 from engaging the bandage 28 that is positioned on the roller 22. The window 56 frictionally engages the exposed portion 38 of the bandage 28 such that the grip 12 is directed away from the window 56 to pull the bandage 28 outwardly and unwind the bandage roll 28.

In use, the bandage 28 is rolled and positioned on the roller 22. The free end 30 of the bandage 28 is positioned around the block 40 such that the adhesive 34 of the bandage 28 faces outwardly from the block 40. The grip 12 is used to direct the dispenser 36 over and around a wound. The bandage 28 is used conventionally to compress and protect the wound.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

1. A bandage dispensing assembly comprising:
a grip;
a mount being attached to said grip;
a roller being rotatably mounted on said mount;
a bandage being wound up to define a bandage roll, said bandage roll being positioned on said roller, a free end of said bandage roll being pulled to unwind said bandage roll from said roller; and
a dispenser being attached to said mount, said bandage being extended through said dispenser defining an exposed portion of said bandage, said dispenser being used to selectively cut off said exposed portion.

2. The bandage dispensing assembly comprising according to claim 1, wherein said mount has a first surface, a second surface and a perimeter edge extending between and being attached to said first surface and said second surface.

3. The bandage dispensing assembly comprising according to claim 2, wherein said roller has a first lateral edge and a second lateral edge, said first lateral edge being rotatably coupled to said first surface of said mount, said second lateral edge extending outwardly from said mount.

4. The bandage dispensing assembly comprising according to claim 3, wherein said bandage comprises a cloth and an adhesive being positioned on said cloth, said adhesive abutting said cloth when said bandage is wound up.

5. The bandage dispensing assembly comprising according to claim 4, wherein said dispenser comprises:
a block being attached to and extending outwardly from said mount, said block guiding said bandage outwardly from said roller;
a cutting member being attached to a top side of said block and being used to cut said bandage; and
a window being attached to and extending outwardly from said block, said window preventing said exposed portion of said bandage from engaging said bandage positioned on said roller.

6. The bandage dispensing assembly comprising according to claim 5, wherein said adhesive of said bandage faces away from said block when pulled outwardly from said roller.

7. The bandage dispensing assembly comprising according to claim 6, wherein said block has a first lateral side, a second lateral side, a front side, a top side and a bottom side, said first lateral side being coupled to said first surface of said mount, said bandage being extended beneath said bottom side of said block and upwardly along said front side of said block to define an exposed portion of said bandage.

8. The bandage dispensing assembly comprising according to claim 7, wherein said cutting member is aligned with said front side of said block, said cutting member having teeth extending outwardly of said front side of said block, said teeth cutting said exposed portion of said bandage to a selected length.

9. The bandage dispensing assembly comprising according to claim 8, wherein said window frictionally engages said exposed portion of said bandage such that said grip is directed away from said window to pull said bandage and unwind said bandage roll.

10. A bandage dispensing assembly comprising:
a grip;
a mount being attached to said grip, said mount having an first surface, a second surface and a perimeter edge extending between and being attached to said first surface and said second surface;
a roller being rotatably mounted on said mount, said roller having a first lateral edge and a second lateral edge, said first lateral edge being rotatably coupled to said first surface of said mount, said second lateral edge extending outwardly from said mount;
a bandage being wound up to define a bandage roll, said bandage roll being positioned on said roller, a free end of said bandage roll being pulled to unwind said bandage roll from said roller, said bandage comprising a cloth and an adhesive being positioned on said cloth, said adhesive abutting said cloth when said bandage is wound up;
a dispenser being attached to said mount, said bandage being extended through said dispenser defining an exposed portion of said bandage, said dispenser being used to selectively cut off said exposed portion, said dispenser comprising:
a block being attached to and extending outwardly from said mount, said block guiding said bandage outwardly from said roller, said adhesive of said bandage facing away from said block when pulled outwardly from said roller, said block having a first lateral side, a second lateral side, a front side, a top side and a bottom side, said first lateral side being coupled to said first surface of said mount;
a cutting member being attached to said top side of said block and being used to cut said bandage, said bandage being extended beneath said bottom side of said block and upwardly along said front side of said block to define an exposed portion of said bandage, said cutting member being aligned with said front side of said block, said cutting member having teeth extending outwardly of said front side of said block, said teeth cutting said exposed portion of said bandage to a selected length; and
a window being attached to and extending outwardly from said block, said window preventing said exposed portion of said bandage from engaging said bandage positioned on said roller, said window frictionally engaging said exposed portion of said bandage such that said grip is directed away from said window to pull said bandage and unwind said bandage roll.
